(19) United States
(12) Patent Application Publication
(10) Pub. No.: US 2002/0187173 A1
(43) Pub. Date: Dec. 12, 2002

(54) Compositions with an optical effect, especially cosmetic compositions

(76) Inventors: Florence L'Alloret, Paris (FR); Maurice Mamane, Choisy Le Roi (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(21) Appl. No.: 10/070,911

(22) PCT Filed: Jan. 14, 2002
(86) PCT No.: PCT/FR02/00122

(30) Foreign Application Priority Data
Jan. 15, 2001 (FR) 0100481

(51) Int. Cl. A61K 7/00

(52) U.S. Cl. 424/401

(57) ABSTRACT

Cosmetic composition comprising an aqueous phase, said aqueous phase comprising at least one compound with an optical effect chosen especially from fillers, pigments, nacres, tensioning agents, matt-effect polymers and mixtures thereof, and a polymer comprising water-soluble units and units having in water a lower critical solution temperature LCST, the heat-induced demixing temperature in aqueous solution of said units with an LCST being from 5 to 40° C. for a concentration by mass in water of from 1% to 25% of said units.

Use of these polymers to eliminate or reduce the tack and to maintain the staying power of a film or deposit obtained from a composition with an optical effect containing them.

The compositions with an optical effect according to the invention may be in the form of emulsions or dispersions and are essentially compositions for topical application and especially cosmetic or pharmaceutical compositions.

The present invention relates to compositions with an optical effect. The invention also relates to the use of specific polymers in particular to improve the staying power of deposits or films obtained from these compositions with an optical effect.

The compositions with an optical effect according to the invention may be in the form of emulsions or dispersions and are essentially compositions for topical application and especially cosmetic compositions.

The technical field of the invention may be defined generally as that of compositions with an optical effect.

The compositions with an optical effect are, for example, in the cosmetic field, make-up or care products providing, after application, a visibly noticeable change in the appearance of the face or the body. Among these compositions, mention is made of foundations, mascaras and products providing coverage or “soft focus”.

The optical properties (visual appearance, coverage, colour or matt effect) of these compositions after application to the skin or to keratin fibres decrease over time, and particularly under hot and humid climatic conditions. The change in the cosmetic deposit or film induced by humidity and heat may be reflected by a loss of transfer-resistance properties, and/or adverse visual changes associated with the poor staying power of this deposit.

In addition, these products or compositions or formulations with an optical effect which are, at room temperature, generally in fluid or gelled form have many limitations regarding their use. Specifically:

fluid formulations are difficult to apply on account of their low viscosity, causing the formulations to run when they are applied;

gelled formulations are obtained with the aid of hydrophilic gelling agents, such as crosslinked polyacrylic acids, in particular the derivatives sold under the name Carbopol , which limit the presentation forms that may be obtained.

In other words, if it is desired at the present time to have available a gel that is pleasant to apply to the skin, use must necessarily be made of a composition which is itself gelled, with all the attendant drawbacks. The variety of textures that may be provided by a composition with an optical effect capable of giving a gel when it is applied to the skin is thus extremely limited.

There is thus a need for a composition with an optical effect, which gives, after application, deposits or films with excellent staying power even under humid and/or hot atmospheric conditions. The said composition that can provide a wide range of textures, especially fluid or gelled textures, in particular at room temperature, allows an easy application.

The aim of the present invention is, inter alia, to satisfy this need.

This aim and others are achieved, in accordance with the invention, by a cosmetic composition comprising an aqueous phase, said aqueous phase comprising at least one compound with an optical effect chosen especially from fillers, pigments, nacres, tensioning agents, matt-effect polymers and mixtures thereof, and a polymer comprising water-soluble units and units having in water a lower critical solution temperature LCST, the heat-induced demixing temperature in aqueous solution of said units with an LCST being from 5 to 40° C. for a concentration by mass in water of from 1% to 25% of said units.

Surprisingly, it has been shown that the cosmetic compositions with an optical effect according to the invention, which comprise the specific polymer as defined above, make it possible to satisfy the needs mentioned above and to satisfy all the requirements mentioned in the foregoing text.

In substance, the properties of a deposit or film obtained from the composition with an optical effect according to the invention including the specific polymer defined above remain constant, do not decrease and do not degrade over time, even when the deposit or film is in contact with a humid and/or hot atmosphere. In addition, the presence of these polymers in the composition does not limit the type of texture of the composition, which may be, for example, fluid or gelled at room temperature.

The films or deposits obtained from the compositions according to the invention, unlike the compositions of the prior art which contain, for example, gelling agents of polyacrylic type, absolutely do not lose their transfer-resistance properties over time, even when exposed to a humid and/or hot atmosphere.

In other words, the deposits obtained after application have a low sensitivity to moisture, and the films or deposits obtained from the compositions of the invention conserve excellent staying power irrespective of the relative humidity and/or the temperature.

The staying power of these deposits and/or films is evaluated, for example, by their “tack”, the reduction of which is a favourable factor with regard to the transfer-resistance and/or staying power properties. Measurements have shown that the tack of the films or deposits obtained from the compositions with an optical effect of the invention remains zero, over a long period, irrespective of the relative humidity and the temperature to which they are exposed and that in all cases, at the worst, the tack of the films or deposits obtained from the compositions of the invention remains less than 0.2 J/m².

The compositions according to the invention may be in any form at a temperature that is below their gel point, for example at room temperature.

That is to say that at a temperature below their gel point, for example at room temperature, the compositions of the invention may have a low or else a high viscosity. This means that “in the jar”, that is to say prior to an increase in temperature generally occurring at the moment they are used, for example when they are applied, generally to the skin (temperature in the region of 32° C.), the range of textures accessible by the compositions of the invention is not limited.

In contrast, the analogous compositions of the prior art can provide only an extremely limited number of textures and must necessarily be in gelled form, if it is desired to obtain a gel, for example during the application.

According to the invention, there is no restriction on the form of the composition at room temperature.

The form of the composition may be varied as desired, and yet, irrespective of this form, for example fluid or gelled, when it is applied, for example to the skin (the temperature of which is generally in the region of 32° C.) which is accompanied by an increase in temperature and/or by an evaporation of the volatile species from the composition, the gelling power of the polymer and gelation will appear.

This gel texture is easy and pleasant to apply.

The gelling power of the polymers according to the invention may thus appear during the application due to an increase in temperature and/or to the evaporation of the volatile species from the composition leading to an increase in concentration.

In other words, the compositions with an optical effect according to the invention are, for example, in fluid form at room temperature, and their viscosity becomes large at a higher temperature, which is that encountered, for example, during application to the skin. Such a gelation, brought about at the temperature of the skin, which is generally in the region of 32° C., allows an easy application of the compositions or formulations according to the invention.

The invention also relates to the use of the polymer as described in the present description, to eliminate or reduce the tack of a film or deposit obtained from a composition with an optical effect containing the said polymer.

Finally, the invention relates to the use of the polymer as described in the present description, to maintain (over time) the staying power of a film or deposit obtained from a composition with an optical effect containing the said polymer.

Such uses of these polymers to improve the properties of the film or deposit obtained from a composition with an optical effect containing this polymer are neither described nor suggested in the prior art. In particular, these films or deposits have no tack or a tack of less than 0.2 J/m² over a long period (for example from 1 to 10 hours), even when they are exposed to a hot and/or humid atmosphere, that is to say, generally, to a temperature from 25° C. to 45° C. and/or to a relative humidity (RH) of from 40 to 95%. The influence exerted by the polymers described in the application, when they are included in a composition with an optical effect, on the tack, the staying power and the transfer resistance has never been described or mentioned in the prior art.

The essential constituent of the compositions according to the invention is a polymer comprising water-soluble units and units having in water a lower critical solution temperature (LCST), also known as “units with an LCST”.

In this respect, it is useful to recall that the expression “Units with an LCST” means units whose solubility in water is modified beyond a certain temperature. These are units with a heat-induced demixing temperature (or cloud point) defining their region of solubility in water. The minimum demixing temperature obtained as a function of the concentration of polymer consisting solely of Units with an LCST is known as the “LCST” (Lower Critical Solution Temperature). For each concentration of polymer with an LCST, a heat-induced demixing temperature is observed. It is higher than the LCST, which is the minimum point of the curve. Below this temperature, the polymer is soluble in water, and above this temperature, the polymer loses its solubility in water.

These units with an LCST of the polymer used in the composition with an optical effect according to the invention correspond to a specific definition which, fundamentally, makes it possible to give the polymer all the advantageous properties described above relating especially to the suppression of the tack and to the improvement in the staying power and in the transfer-resistance properties of films or deposits obtained from these compositions.

The units with an LCST of the polymer have, according to the invention, a heat-induced demixing temperature of from 5 to 40° C. for a concentration by mass in water of from 1% to 25% by weight of the said units with an LCST.

Preferably, the heat-induced demixing temperature in aqueous solution of the Units with an LCST of the polymer is from 10 to 35° C. for a concentration by mass in water of from 1% to 25% of the said units with an LCST.

The polymer having the structure described above with water-soluble units and specific Units with an LCST defined above has in aqueous solution gelation properties beyond a critical temperature, or heat-induced gelling properties.

These heat-induced gelling properties observed beyond the demixing temperature of the chains with an LCST are described in the prior art, especially in documents [1], [2] and [3]. They are due to the combination of the chains with an LCST within hydrophobic microdomains beyond their demixing temperature, thus forming crosslinking nodes between the main chains.

These gelling properties are observed when the polymer concentration is sufficient to allow interactions between units with an LCST borne by different macromolecules. The minimum concentration required, known as the “critical aggregation concentration”, or CAC, is evaluated by rheological measurements: it is the concentration at and above which the viscosity of an aqueous solution of the polymers of the invention becomes higher than the viscosity of a solution of the equivalent polymer not comprising Chains with an LCST.

Beyond the CAC, the polymers of the invention have gelling properties when the temperature becomes higher than a critical value, known as the “gel point”, or T_{gel}. According to the literature data, there is good agreement between T_{gel} and the demixing temperature of the chains with an LCST, under the same concentration conditions. The gel point of an aqueous solution of a polymer of the invention is determined by Theological measurements: it is the temperature at and above which the viscosity of a solution of a polymer of the invention becomes higher than the viscosity of a solution of the equivalent polymer not comprising chains with an LCST.

The polymers of the invention are characterized by a specific gel point generally of from 5 to 40° C. and preferably from 10 to 35° C., for a concentration by mass in water of from 2% to 50% by weight.

It should be noted that this concentration is generally not that of the compositions of the invention, but that it may be achieved by means of the evaporation of the volatile species from the composition, for example during the application, thus bringing about the appearance of the gelling power of the polymer.

As a function of this specific gel point under the conditions of use, for example during application to the skin, the compositions of the invention have a low or high viscosity and thus a wide variety of forms at room temperature, and their favourable effects on the deposit or film obtained, when they are applied, for example to the skin, are still maintained.

Polymers comprising, in the manner of those used in the compositions of the invention, water-soluble units and units with an LCST and having heat-induced gelling properties observed beyond the demixing temperature of the chains with an LCST are described in the documents already mentioned above.

Document [1] relates to the reversible heat-induced thickening of aqueous solutions of copolymers comprising a water-soluble backbone of polyacrylic acid with poly(ethylene oxide) (PEO) grafts.

Document [2] relates to the heat-induced thickening behaviour in aqueous solution of polymers comprising a 2-acrylamido-2-methylpropanesulphonic acid (AMPS) backbone and poly(ethylene oxide) side chains.

Similarly, document [3] describes the reversible heat-induced association of copolymers with a polyacrylic water-soluble backbone or a backbone based on AMPS with PEO grafts.

Polymers, such as those mentioned in documents [1], [2] and [3], find their use in particular in the petroleum industry.

Thus, document [4] describes thermoviscosifying polymers with a water-soluble backbone comprising segments with an LCST, or bearing side chains with an LCST, which may be used especially as thickeners, constituents of drilling fluids or other fluids, and industrial cleaning fluids.

Document [5] describes polymers similar to those of document [4] and their use as anti-sedimentation agents for suspensions, possibly in cosmetic preparations.

It should be noted that none of the documents [1] to [5] mentions the incorporation of polymers into compositions with an optical effect.

Document [6] also describes copolymers comprising a backbone consisting of pH-sensitive units, for example polyacrylic units, and heat-sensitive units, grafted onto this backbone. These copolymers have heat-induced gelling properties and they are used for the liberation and controlled release of active principles or pharmaceutical agents, and possibly cosmetic agents, by topical application.

The heat-sensitive units in the copolymers of this document are not the specific units with an LCST of the polymers of the invention. Furthermore, the polymers according to document [6] are characterized by the extremely inconvenient opacity of the heat-induced gels, which is not the case for the polymers used in the compositions with an optical effect of the invention.

In point of fact, the polymer in the said document is fundamentally different from that of the invention since it has overall for the entire polymer an LCST in the temperature range from 20 to 40° C., rather than a gel point.

Documents [7] and [8] describe reversible-gelling polymer systems, comprising a sensitive component capable of aggregation, in response to a change in an external “stimulus”, and a structural component. The external stimulus may be, for example, the temperature.

The component that is sensitive to the external stimulus is fundamentally different from the units with an LCST of the present application. Specifically, these components that are sensitive to the external stimulus in fact consist of at least one hydrophilic fragment and one hydrophobic fragment. Thus, the sensitive component may be a block copolymer, such as a “poloxamer”, for example a Pluronic®, which is a block polymer of ethylene oxide (soluble) and of propylene oxide (insoluble); such a block copolymer aggregates microscopically beyond a critical temperature not corresponding to an LCST.

Document [7] relates more particularly to a polymer network formed from a water-soluble polyacrylic backbone and a Pluronic® sensitive component, which is interlocked in the said backbone, without covalent bonding; this network thus has a particular structure that has nothing in common with the polymer used in the invention. On the other hand, in document [8], it is a mattr of polymers with covalent bonds.

These polymers have heat-induced gelling properties and they may be used in the pharmaceutical field for the delivery of medicinal products and in many other fields, including the cosmetics field. As regards the cited topical and cosmetic uses, only examples of emulsions, such as antisun products and care products, but not compositions with an optical effect, are mentioned. They all contain a neutral, anionic or cationic surfactant, along with the polymer acting as gelling agent.

The use of the polymers in particular compositions with an optical effect is never mentioned, nor are their properties in film form, and even less so their favourable effect, inter alia, on the staying power and the tack of these films. This is likewise the case for the stabilizing and texture effects obtained in the invention.

In these formulations, the sensitive component of the polymer system has a behaviour that is different from that of the units with an LCST, such as those of the polymer of the invention, during heating. Thus, when the said sensitive component (for example poloxamer) is heated to about 30-40° C., it shows a temperature of micellization, that is to say an aggregation at the microscopic level, and then, when it is heated further, an LCST temperature that is very much higher. This LCST corresponds to an aggregation at the macroscopic level between the chains with an LCST. This difference in behaviour between the polymers comprising units with an LCST off the invention and the polymer systems using poloxamers is explained in WO-A-97/00275 [8] on pages 16 and 17, where it stated that the gelation and the LCST are observed at temperatures which differ by about 70° C., the gel point corresponding to the micellization temperature of the sensitive component. In addition, it is not possible, on account of the synthesis used in document [8], to fully control the structure and properties of the final polymer obtained, as is the case in the compositions of the invention.

Cosmetic compositions using a reversible heat-induced gelling polymer system, comprising polyacrylic acid and a poloxamer as in documents [7] and [8], are also known from document [9]. Once again, the polymer system of these documents is fundamentally different from that used in the compositions of the invention, since it aggregates at the microscopic level beyond a critical temperature not corresponding to an LCST.

WO-A-00/35961 [10] describes the preparation of polymers with heat-induced thickening properties by emulsion polymerization and the use of these polymers in pharmaceutical and cosmetic compositions. These polymers may be copolymers containing water-soluble units and units with an LCST based on alkylene oxide. It is envisaged to add nonionic surfactants to the polymers to reinforce their heat-induced thickening properties.

It emerges from the foregoing text that the use in compositions with an optical effect of the polymers according to the invention, containing specific units with an LCST, is never described or suggested in the prior art.

Lesser still, no description is given of the properties in the form of films of such compositions or of all the specific effects and advantages regarding the properties of these films associated with the use of the specific polymers described in the invention.

The polymers used in the invention may be block polymers or grafted polymers, which comprise, on the one hand, water-soluble units and, on the other hand, units with an LCST as defined above.

It is pointed out that, in the present text, the water-soluble units or the units with an LCST of the polymers used according to the invention are defined as not including the groups linking together, on the one hand, the said water-soluble units and, on the other hand, the said units with an LCST.

The said linking groups are derived from the reaction, during the preparation of the polymer, of the reactive sites borne, on the one hand, by the precursors of the said water-soluble units and, on the other hand, by the precursors of the said units with an LCST.

The polymers used in the context of the invention may thus be block polymers comprising, for example, blocks consisting of water-soluble units alternating with blocks with an LCST.

These polymers may also be in the form of grafted polymers whose backbone is formed from water-soluble units, the said backbone bearing grafts consisting of units with an LCST.

The said polymers may be partially crosslinked.

The expression “water-soluble units” generally means that these units are units that are soluble in water, at a temperature of from 5 to 80° C., to a proportion of at least 10 g/l and preferably of at least 20 g/l.

However, the expression “water-soluble units” also means units not necessarily having the solubility mentioned above, but which, in aqueous solution at 1% by weight, from 5 to 80° C., allow the production of a macroscopically homogeneous and transparent solution, that is to say a solution having a maximum light transmittance value, irrespective of the wavelength of between 400 and 800 nm, through a sample 1 cm thick, of at least 85% and preferably of at least 90%.

These water-soluble units do not have a heat-induced demixing temperature of LCST type.

These water-soluble units are totally or partially obtainable by polymerization, especially free-radical polymerization, or by polycondensation, or alternatively consist totally or partially of existing natural polymers or modified natural polymers.

By way of example, the water-soluble units are totally or partially obtainable by polymerization, especially free-radical polymerization, of at least one monomer chosen from the following monomers:

(meth)acrylic acid;

vinyl monomers of formula (I) below:

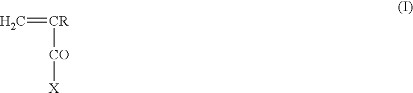

in which:

R is chosen from H, —CH₃, —C₂H₅ or —C₃H₇; and

X is chosen from:

alkyl oxides of —OR′ type in which R′ is a linear or branched, saturated or unsaturated hydrocarbon-based radical containing from 1 to 6 carbon atoms, optionally substituted with at least one halogen atom (iodine, bromine, chlorine or fluorine); a sulphonic (—SO₃⁻), sulphate (—SO₄⁻), phosphate (—PO₄H₂); hydroxyl (—OH); primary amine (—NH₂); secondary amine (—NHR₁), tertiary amine (—NR₁R₂) or quaternary amine (—N⁺R₁R₂R₃) group with R₁, R₂ and R₃ being, independently of each other, a linear or branched, saturated or unsaturated hydrocarbon-based radical containing 1 to 6 carbon atoms, with the proviso that the sum of the carbon atoms of R′+R₁+R₂+R₃ does not exceed 7; and

—NH₂, —NHR₄ and —NR₄R₅ groups in which R₄ and R₅ are, independently of each other, linear or branched, saturated or unsaturated hydrocarbon-based radicals containing 1 to 6 carbon atoms, with the proviso that the total number of carbon atoms of R₄+R₅ does not exceed 7, the said R₄ and R₅ optionally being substituted with a halogen atom (iodine, bromine, chlorine or fluorine); a hydroxyl (—OH); sulphonic (—SO₃⁻); sulphate (—SO₄⁻); phosphate (—PO₄H₂); primary amine (—NH₂); secondary amine (—NHR₁), tertiary amine (—NR₁R₂) and/or quaternary amine (—N⁺R₁R₂R₃) group with R₁, R₂ and R₃ being, independently of each other, a linear or branched, saturated or unsaturated hydrocarbon-based radical containing 1 to 6 carbon atoms, with the proviso that the sum of the carbon atoms of R₄+R₅+R₁+R₂+R₃ does not exceed 7;

maleic anhydride;

itaconic acid;

vinyl alcohol of formula CH₂═CHOH;

vinyl acetate of formula CH₂═CH—OCOCH₃;

N-vinyllactams such as N-vinylpyrrolidone, N-vinylcaprolactam and N-butyrolactam;

vinyl ethers of formula CH₂═CHOR₆ in which R₆ is a linear or branched, saturated or unsaturated hydrocarbon-based radical containing from 1 to 6 carbons;

water-soluble styrene derivatives, especially styrene sulphonate;

dimethyldiallylammonium chloride; and

vinylacetamide.

The polycondensates and natural polymers or modified natural polymers which may constitute all or part of the water-soluble units are chosen from one or more of the following components:

water-soluble polyurethanes,

xanthan gum, especially the product sold under the names Keltrol T and Keltrol SF by Kelco; or Rhodigel SM and Rhodigel 200 from Rhodia;

alginates (Kelcosol from Monsanto) and derivatives thereof such as propylene glycol alginate (Kelcoloid LVF from Kelco);

cellulose derivatives and especially carboxymethylcellulose (Aquasorb A500, Hercules), hydroxypropylcellulose, hydroxyethylcellulose and quaternized hydroxyethylcellulose;

galactomannans and derivatives thereof, such as Konjac gum, guar gum, hydroxypropylguar, hydroxypropylguar modified with sodium methyl-carboxylate groups (Jaguar XC97-1, Rhodia), hydroxypropyltrimethylammonium guar chloride.

Mention may also be made of polyethyleneimine.

The water-soluble units preferably have a molar mass ranging from 1000 g/mol to 5 000 000 g/mol when they constitute the water-soluble backbone of a grafted polymer.

These water-soluble units preferably have a molar mass ranging from 500 g/mol to 100 000 g/mol when they constitute a block of a multiblock polymer.

The units with an LCST of the polymers used in the invention may be defined as being units whose water solubility is modified beyond a certain temperature. They are units with a heat-induced demixing temperature (or cloud point) defining their region of solubility in water. The minimum demixing temperature obtained as a function of the polymer concentration is referred to as the “LCST” (Lower Critical Solution Temperature). For each polymer concentration, a heat-induced demixing temperature is observed; it is higher than the LCST, which is the minimum point of the curve. Below this temperature, the polymer constituting the unit with an LCST is soluble in water; above this temperature, the polymer constituting the unit with an LCST loses its solubility in water.

Some of these polymers with an LCST are especially described in the articles by Taylor et al., Journal of Polymer Science, part A: Polymer Chemistry, 1975, 13, 2551 [11]; by J. Bailey et al., Journal of Applied Polymer Science, 1959, 1,56 [12]; and by Heskins et al., Journal of Macromolecular Science, Chemistry A2, 1968, 1441 [13].

The expression “soluble in water at a temperature T” means that the units have a solubility at T of at least 1 g/l and preferably of at least 2 g/l.

The measurement of the LCST may be performed visually: the temperature at which the cloud point of the aqueous solution appears is determined; this cloud point is reflected by the opacification of the solution, or the loss of transparency.

In general, a transparent composition will have a maximum light transmittance value, irrespective of the wavelength of between 400 and 800 nm, through a sample 1 cm thick, of at least 85% and preferably of at least 90%.

The transmittance may be measured by placing a sample 1 cm thick in the light beam of a spectrophotometer working at the wavelengths of the light spectrum.

The units with an LCST of the polymers used in the invention may consist of one or more polymers chosen from the following polymers:

polyethers such as polyethylene oxide (PEO), polypropylene oxide (PPO) or random copolymers of ethylene oxide (EO) and of propylene oxide (PO),

polyvinyl methyl ether,

polymeric and copolymeric N-substituted acrylamide derivatives containing units with an LCST, such as poly-N-isopropylacrylamide (Nipam) and poly-N-ethylacrylamide; and

polyvinylcaprolactam and vinylcaprolactam copolymers.

Preferably, the units with an LCST consist of polypropylene oxide (PPO)ₙ where n is an integer from 10 to 50, or of random copolymers of ethylene oxide (EO) and of propylene oxide (PO), represented by the formula:

(EO)ₘ(PO)ₙ

in which m is an integer ranging from 1 to 40 and preferably from 2 to 20, and n is an integer ranging from 10 to 60 and preferably from 20 to 50.

Preferably, the molar mass of these units with an LCST is from 500 to 5300 g/mol and more preferably from 1500 to 4000 g/mol.

It has been found that the random distribution of the EO and PO units is reflected by the existence of a lower critical solution temperature, beyond which a macroscopic phase separation is observed. This behaviour is different from that of block (EO) (PO) copolymers, which form micelles beyond a critical temperature known as the micellization temperature (microscopic aggregation).

The units with an LCST may thus especially be derived from aminated, especially monoamino, diamino or triamino, random copolymers of ethylene oxide and of propylene oxide, which are the copolymers that are the precursors of these units with an LCST. These precursors bear reactive sites, in this case amino groups, reacting with the reactive sites of the water-soluble precursors, for example carboxyl groups, to give the final polymer used in the invention. In the final polymer, the water-soluble units are linked to the units with an LCST via linking groups derived from the reaction of the reactive sites or groups borne, respectively, by the presursors of the units with an LCST and the precursors of the water-soluble units. These linking groups will be, for example, amide, ester, ether or urethane groups.

Among the commercially available precursor polymers from which the units with an LCST are derived, mention may be made of the copolymers sold under the name Jeffamine by Huntsman, and especially Jeffamine XTJ-507 (M-2005), Jeffamine B-2000 and Jeffamine XTJ-509 (or T-3000).

The units with an LCST may also be derived from random EO/PO precursor copolymers containing OH end groups, such as those sold under the name Polyglycols P41 and B11 by Clariant.

Polymeric and copolymeric N-substituted acrylamide derivatives containing units with an LCST, and also polyvinylcaprolactam and vinylcaprolactam copolymers may also be used in the invention as units with an LCST.

As examples of polymeric and copolymeric N-substituted acrylamide derivatives containing units with an LCST, mention may be made of poly-N-isopropylacrylamide, poly-N-ethylacrylamide and copolymers of N-isopropylacrylamide (or of N-ethylacrylamide) and of a vinyl monomer chosen from the monomers having the formula (I) given above, maleic anhydride, itaconic acid, vinylpyrrolidone, styrene and its derivatives, dimethyldiallylammonium chloride, vinylacetamide, vinyl ethers and vinyl acetate derivatives.

The molar mass of these polymers is preferably from 1000 g/mol to 50 000 g/mol and preferably from 2000 to 50 000 g/mol.

These polymers may be synthesized by free-radical polymerization using a pair of initiators such as aminoethanethiol hydrochloride, in the presence of potassium persulphate, so as to obtain precursor oligomers with a reactive amino end group.

As examples of vinylcaprolactam copolymers, mention may be made of copolymers of vinylcaprolactam and of a vinyl monomer of formula (I) given above, or of a monomer chosen from maleic anhydride, itaconic acid, vinylpyrrolidone, styrene and its derivatives, dimethyldiallylammonium chloride, vinylacetamide, vinyl alcohol, vinyl acetate, vinyl ethers and vinyl acetate derivatives.

The molar mass of these vinylcaprolactam polymers or copolymers is generally from 1000 g/mol to 500 000 g/mol and preferably from 2000 to 50 000 g/mol.

These compounds may be synthesized by free-radical polymerization using a pair of initiators such as aminoethanethiol hydrochloride, in the presence of potassium persulphate, so as to obtain precursor oligomers of the units with an LCST with a reactive amino end group.

The proportion by mass of the units with an LCST in the final polymer is preferably from 5% to 70%, especially from 20% to 65% and particularly from 30% to 60% by weight relative to the final polymer.

As defined above, the heat-induced demixing temperature of the said units with an LCST of the polymer used in the invention is from 5 to 40° C. and preferably from 10 to 35° C., for a concentration by mass in water of from 1% to 25% by weight of the said units with an LCST.

The polymers used in the context of the invention may be readily prepared by a person skilled in the art on the basis of his general knowledge, using grafting, copolymerization or coupling reaction processes.

When the final polymer is in the form of a grafted polymer, especially having a water-soluble backbone with side chains or grafts with an LCST, it is possible to prepare it by grafting precursors of the units with an LCST containing at least one reactive end group or reactive site, especially an amino end group or site, onto a water-soluble precursor polymer forming the backbone, bearing at least 10% (on a molar basis) of reactive groups such as carboxylic acid functions. This reaction may be carried out in the presence of a carbodiimide such as dicyclohexylcarbodiimide or 1-(3-dimethylaminopropyl)-3-ethylcarbodiimide hydrochloride, in a solvent such as N-methylpyrrolidone or water.

Another possibility for preparing grafted polymers consists in copolymerizing, for example, an macromonomer with an LCST (chain with an LCST described above with a vinyl end group) and a water-soluble vinyl monomer such as acrylic acid or vinyl monomers of formula (I).

When the final polymer is in the form of a block polymer, it is possible to prepare it by coupling between precursors of the water-soluble units and precursors of the units with an LCST, these precursors having complementary reactive sites at each end.

In the case of grafting processes and coupling processes, the reactive sites of the precursors of the units with an LCST may be amine functions, especially monoamine, diamine or triamine functions, and OH functions. In this case, the reactive sites of the precursors of the water-soluble units may be carboxylic acid functions. The groups linking the water-soluble units and the units with an LCST will thus be, for example, amide groups or ester groups.

As has been seen previously, the compositions with an optical effect of the invention comprise an aqueous phase.

According to the invention, the aqueous phase comprises a polymer comprising water-soluble units and units with an LCST, as defined above. Generally, the concentration by mass of polymer in the aqueous phase is from 0.1 to 20% and preferably from 0.5 to 10%.

In addition, the aqueous phase of the composition according to the invention comprises at least one compound with an optical effect. In the present invention, the expression “compound with an optical effect” means a compound which gives the composition optical properties (for example a matt effect, colour or coverage) when applied to the skin.

This compound with an optical effect may be chosen especially from fillers, pigments, nacres, tensioning agents and matt-effect polymers, and mixtures thereof.

The term “fillers” should be understood as meaning colourless or white, mineral or synthetic, lamellar or non-lamellar particles intended to give body or rigidity to the composition and/or softness, a matt effect and uniformity to the make-up. Fillers that may especially be mentioned are talc, mica, silica, boron nitride, bismuth oxychloride, kaolin, Nylon powders such as Nylon-12 (Orgasol sold by the company Atochem), polyethylene powders, Teflon (tetrafluoroethylene polymer powders), polyurethane powders, polystyrene powders, polyester powders, optionally modified starch, copolymer microspheres, such as those sold under the name Expancel by the company Nobel Industrie, microsponges, for instance Polytrap sold by the company Dow Corning, silicone resin microbeads such as those sold by the company Toshiba under the name Tospearl, precipitated calcium carbonate, magnesium carbonate, magnesium hydrocarbonate, hydroxyapatite, hollow silica microspheres (Silica Beads from the company Maprecos), glass or ceramic microcapsules, metal soaps derived from organic carboxylic acids containing from 8 to 22 carbon atoms and preferably from 12 to 18 carbon atoms, for example zinc, magnesium or lithium stearate, zinc laurate or magnesium myristate, and mixtures thereof.

The term “pigments” should be understood as meaning white or coloured, mineral or organic particles, insoluble in the medium, that are intended to colour and/or opacify the composition. They may be white or coloured, mineral and/or organic, and of standard or nanometric size. Among the mineral pigments and nanopigments that may be mentioned are titanium dioxide, zirconium dioxide or cerium dioxide, and also zinc oxide, iron oxide or chromium oxide, nanotitaniums (titanium dioxide nanopigments), nanozincs (zinc oxide nanopigments) and ferric blue. Among the organic pigments that may be mentioned are carbon black and lakes, for instance calcium, barium, aluminium or zirconium salts, or salts of acidic dyes such as halo acid dyes, azo dyes or anthraquinone dyes.

The term “nacres” should be understood as meaning iridescent particles that reflect light. Among the nacres that may be envisaged, mention may be made of natural mother-of-pearl, mica coated with titanium oxide, with iron oxide, with natural pigment or with bismuth oxychloride, and also coloured titanium mica.

The term “tensioning agent” should be understood as meaning compounds capable of having a tensioning effect, that is to say capable of making the skin taut and, by this tensioning effect, smoothing out the skin and causing the wrinkles and fine lines therein to reduce or disappear immediately. Tensioning agents that may be mentioned include polymers of natural origin as described, for example, in document EP-912 165; synthetic polymers in latex and pseudolatex form, such as those described in document EP-A-944 381; dendritic polymers as described in document EP-A-987 016; “starburst” polymers as described in document EP-A-1 043 345; mixed silicates; and mixtures thereof.

The expression “polymer of natural origin” means polymers of plant origin, polymers derived from integuments, egg proteins and latices of natural origin. These polymers are preferably hydrophilic.

As polymers of plant origin, mention may especially be made of proteins and protein hydrolysates, and more particularly extracts of cereals, of leguminous plants and of oil-bearing plants, such as extracts of corn, of rye, of wheat, of buckwheat, of sesame, of spelt, of pea, of bean, of lentil, of soybean and of lupin. Suitable proteins that may be mentioned, for example, include the extract of soybean protein sold by the company ISD under the name “Profam 972” and the protein fraction of white lupin.

Examples of polymers derived from integuments that may be mentioned include chitin and its derivatives, especially chitosan which is a deacetylated derivative of chitin, and also chitosan derivatives such as hydroxypropylchitosan, the succinylated derivative of chitosan, chitosan lactate, chitosan glutamate and carboxymethylchitosan succinimide; keratin derivatives such as keratin hydrolysates and sulphonic keratins.

Egg proteins that may be mentioned include egg albumin.

Examples of latices of natural origin that may be mentioned include shellac resin, sandarac gum, dammar resins, elemi gums, copal resins and cellulose derivatives.

The synthetic polymers in the form of a latex or a pseudolatex may be of polycondensate type or of free-radical type. Mention may be made especially of dispersions of polyesterpolyurethane and of polyetherpolyurethane, sold under the names “Sancure 2060” (polyesterpolyurethane), “Sancure 2255” (polyesterpolyurethane), “Sancure 815” (polyesterpolyurethane), “Sancure 878” (polyetherpolyurethane) and “Sancure 861” (polyetherpolyurethane) by the company Sanncor, under the names “Neorez R974” (polyesterpolyurethane), “Neorez R981” (polyesterpolyurethane) and “Neorez R970” (polyetherpolyurethane) by the company ICI, and the acrylic copolymer dispersion sold under the name “Neocryl XK-90” by the company Zeneca.

Dendritic polymers that may especially be mentioned are those of polyester type with hydroxyl end functions and optionally bearing chain-terminating groups, sold by the company Perstorp, such as the dendritic polyester obtained by polycondensation of dimethylolpropionic acid with trimethylolpropane, free of chain-terminating agents, sold under the name Boltorn H40 TMP Core; the dendritic polyester obtained by polycondensation of dimethylolpropionic acid with polyoxyethylenated pentaerythritol (5 units of EO on each hydroxyl function), 50% of the hydroxyl functions of which are esterified with capric/caprylic acid (technical name: esterified HBP 3G) and the dendritic polyester obtained by polycondensation of dimethylolpropionic acid with polyoxyethylenated pentaerythritol (5 units of EO on each hydroxyl function), free of chain-terminating agents (technical name: HBP Polyol 3G).

Mixed silicates that may be used in the present invention include all silicates of natural or synthetic origin containing several types of cation chosen from alkali metals (for example Na, Li or K) or alkaline-earth metals (for example Be, Mg or Ca) and transition metals, and more particularly laponites, which are silicates of magnesium, lithium and sodium with a layer structure similar to that of montmorillonites. Laponites that may be mentioned especially include the product sold, for example, under the name Laponite XLS or Laponite XLG by the company Southern Clay Products.

The expression “matt-effect polymers” means herein any polymer in solution, in dispersion or in the form of particles, which prevents the skin from shining and which unifies the complexion. Examples which may be mentioned are silicone elastomers; resin particles; and mixtures thereof.

Examples of silicone elastomers that may be mentioned include the products sold under the names “KSG” by the company Shin-Etsu, under the names “Trefil”, “BY29” or “EPSX” by the company Dow Corning or under the names “Gransil” by the company Grant Industries.

Examples of resin particles that may be mentioned include those of melamine-formaldehyde or of urea-formaldehyde resin, described in document EP-A-1 046 388.

The concentration by mass in the aqueous phase of these fillers and/or pigments and/or nacres is generally from 0.1 to 20% and preferably from 0.2 to 15% by weight relative to the total weight of the composition.

The composition with an optical effect according to the invention may comprise essentially only the said aqueous phase, and it will then be in the form of a dispersion of fillers and/or pigments and/or nacres containing the polymer defined above.

However, generally, the composition with an optical effect according to the invention is in the form of an oil-in-water emulsion comprising the said aqueous phase and an oily phase dispersed, preferably uniformly, in the said aqueous phase comprising a polymer comprising water-soluble units and units with an LCST as defined above. The concentration by mass of polymer in this aqueous phase of the emulsion is that given above.

The oily phase of the emulsion comprises at least one fatty substance and preferably at least one oil.

As oils which can be used in the composition of the invention, mention may be made for example of:

hydrocarbon-based oils of animal origin, such as perhydrosqualene;

hydrocarbon-based oils of plant origin, such as liquid triglycerides of fatty acids of 4 to 10 carbon atoms, such as heptanoic or octanoic acid triglycerides or alternatively, for example, sunflower oil, corn oil, soybean oil, marrow oil, grapeseed oil, sesame oil, hazelnut oil, apricot oil, macadamia oil, arara oil, castor oil, avocado oil, caprylic/capric acid triglycerides such as those sold by the company Stearineries Dubois or those sold under the names Miglyol 810, 812 and 818 by the company Dynamit Nobel, jojoba oil or karite butter;

synthetic esters and ethers in particular of fatty acids, such as the oils of formulae R¹COOR² and R¹OR² in which R¹ represents a fatty acid residue containing from 8 to 29 carbon atoms and R² represents a branched or unbranched hydrocarbon-based chain containing from 3 to 30 carbon atoms, such as, for example, purcellin oil, isononyl isononanoate, isopropyl myristate, 2-ethylhexyl palmitate, 2-octyldodecyl stearate, 2-octyldodecyl erucate or isostearyl isostearate; hydroxylated esters such as isostearyl lactate, octyl hydroxystearate, octyldodecyl hydroxystearate, diisostearyl malate, triisocetyl citrate, and fatty alcohol heptanoates, octanoates and decanoates; polyol esters such as propylene glycol dioctanoate, neopentyl glycol diheptanoate and diethylene glycol diisononanoate; and pentaerythritol esters such as pentaerythrityl tetraisostearate;

linear or branched hydrocarbons of mineral or synthetic origin, such as volatile or non-volatile liquid paraffins and derivatives thereof, petroleum jelly, polydecenes or hydrogenated polyisobutene such as parleam oil;

natural or synthetic essential oils such as, for example, eucalyptus oil, hybrid lavender oil, lavender oil, vetiver oil, Litsea cubeba oil, lemon oil, sandalwood oil, rosemary oil, camomile oil, savory oil, nutmeg oil, cinnamon oil, hyssop oil, caraway oil, orange oil, geraniol oil, cade oil and bergamot oil;

fatty alcohols containing from 8 to 26 carbon atoms, such as cetyl alcohol, stearyl alcohol, and the mixture thereof (cetylstearyl alcohol), octyldodecanol, 2-butyloctanol, 2-hexyldecanol, 2-undecylpentadecanol, oleyl alcohol or linoleyl alcohol;

partially hydrocarbon-based and/or silicone-based fluoro oils such as those described in document JP-A-2-295 912;

silicone oils such as volatile or non-volatile polydimethylsiloxanes (PDMSs) containing a linear or cyclic silicone chain, which are liquid or pasty at room temperature, in particular cyclopolydimethylsiloxanes (cyclomethicones) such as cyclohexasiloxane; polydimethylsiloxanes comprising alkyl, alkoxy or phenyl groups, pendent or at the end of a silicone chain, these groups containing from 2 to 24 carbon atoms; phenylsilicones such as phenyl trimethicones, phenyl dimethicones, phenyltrimethylsiloxydiphenylsiloxanes, diphenyl dimethicones, diphenylmethyldiphenyltrisiloxanes, 2-phenylethyl trimethylsiloxysilicates and polymethylphenylsiloxanes;

mixtures thereof.

The term “hydrocarbon-based oil” in the list of abovementioned oils embraces any oil comprising predominantly carbon and hydrogen atoms, and optionally ester, ether, fluoro, carboxylic acid and/or alcohol groups.

The other fatty substances which may be present in the oily phase are, for example, fatty acids containing from 8 to 30 carbon atoms, for instance stearic acid, lauric acid, palmitic acid and oleic acid; waxes, for example lanolin, beeswax, carnauba wax, candelilla wax, paraffin wax, lignite wax or microcrystalline waxes, ceresine or ozokerite, synthetic waxes, for instance polyethylene waxes and Fischer-Tropsch waxes; gums such as silicone gums (dimethiconol); silicone resins such as trifluoromethyl-Cl-4-alkyldimethicone and trifluoropropyldimethicone.

These fatty substances may be chosen in a varied manner by a person skilled in the art in order to prepare a composition having the desired properties, for example consistency or texture properties.

The amount of oily phase in the emulsion may range, for example, from 0.01% to 50% by weight and preferably from 0.1% to 30% by weight relative to the total weight of the composition.

When the composition is in the form of emulsions, it may advantageously contain an emulsifying surfactant.

Emulsifying surfactants that may especially be mentioned include nonionic emulsifiers, and, for example, the adducts of 1 to 200 mol of ethylene oxide or of propylene oxide with partial esters of polyols containing 2 to 16 carbon atoms and of fatty acids containing 12 to 22 carbon atoms, for instance fatty acid esters of polyethylene glycol such as PEG-100 stearate, PEG-50 stearate and PEG-40 stearate; fatty acid esters of polyols such as glyceryl stearate, sorbitan tristearate and oxyethylenated sorbitan stearates sold under the trade names Tween® 20 or Tween® 60, and sugar esters, for instance sucrose stearate, and mixtures thereof.

The aqueous phase may also optionally comprise a gelling agent in a concentration by mass of from 0.01 to 5% and preferably from 0.01 to 3% by weight relative to the total weight of the composition.

In the compositions with an optical effect of the invention, the aqueous phase preferably consists of a physiologically acceptable medium allowing a topical application and especially a cosmetic application.

In the present patent application, the expression “physiologically acceptable medium” means a medium that is compatible with all keratin materials such as the skin, including the scalp, the nails, mucous membranes, the eyes and the hair or any other area of body skin.

The physiologically acceptable medium of the compositions with an optical effect of the invention comprises water. The amount of water may range from 30 to 99.98% by weight and preferably from 40 to 95% by weight relative to the total weight of the composition.

The water used may be, besides water, a floral water such as cornflower water, a mineral water such as eau de Vittel, eau de Lucas or eau de la Roche Posay and/or a spring water.

The physiologically acceptable medium may contain, besides water, one or more solvents chosen from lower alcohols containing from 1 to 8 carbon atoms, such as ethanol; polyols such as glycerol; glycols, for instance butylene glycol, isoprene glycol, propylene glycol, polyethylene glycols such as PEG-8; sorbitol; sugars such as glucose, fructose, maltose, lactose and sucrose; and mixtures thereof. The amount of solvent(s) may range from 0.5 to 30% by weight and preferably from 5 to 20% by weight relative to the total weight of the aqueous phase.

The compositions with an optical effect of the invention may also contain adjuvants commonly used in cosmetics and dermatology, such as hydrophilic or lipophilic active agents, preserving agents, gelling agents, plasticizers, antioxidants, fragrances, odour absorbers, antifoams, UV screening agents, sequestering agents (EDTA), acidic or basic pH adjusters or buffers, and colorants.

The amounts of these various additives are those conventionally used in the fields under consideration, and, for example, from 0.01 to 20% of the total weight of the composition. Needless to say, a person skilled in the art will take care to select the optional compound(s) to be added to the foaming compositions according to the invention such that the advantageous properties intrinsically associated with these compositions are not, or are not substantially, adversely affected by the addition envisaged.

As has already been mentioned above, a gelling agent may be added to the compositions of the invention in order to adjust the texture of the composition and to gain access to a wide range of textures from a milk to a cream. As already mentioned above, the compositions according to the invention can at low temperature, for example at room temperature, “in the jar”, provide any desirable texture. There is no restriction as regards the texture that the composition can have, before application. In particular, the composition does not necessarily need to contain a gelling agent in order to obtain a gelled texture when applied.

Specifically, by virtue of the specific polymer included in the composition of the invention, a gelled and stable texture is obtained during the increase in temperature taking place, for example, when the composition is applied, especially to the skin, irrespective of the texture or the form of the composition with an optical effect “in the jar” before application. Thus, a gelling agent will be included in the composition only if it is desired for the said composition to have a gelled appearance, this appearance being, according to the invention, only one particular appearance among the multitude of appearances, textures and forms that the composition with an optical effect may have.

The gelling agents that may be used may be hydrophilic gelling agents. Examples of hydrophilic gelling agents that may be mentioned in particular are carboxyvinyl polymers (carbomer), acrylic copolymers such as acrylate/alkylacrylate copolymers, polyacrylamides, polysaccharides, natural gums and clays.

The compositions with an optical effect of the invention may especially be in the form of a cosmetic, make-up or care composition that may be applied to the skin, including the scalp, the nails, the hair, the eyelashes, the eyebrows, the eyes, mucous membranes and semi-mucous membranes, and any other area of body or facial skin.

These compositions with an optical effect can generally be defined as products such as make-up or care products that provide, after application, a change in the appearance of the face or the body that is visually noticeable to the naked eye. The term “appearance” means not only the colour, but also the surface appearance, the relief, etc.

Examples of such compositions that may be mentioned include foundations, face powders, eyeshadows, mascaras, products providing coverage or “soft focus”, antisun products and hair products.

It is recalled that a composition providing “soft focus” is a composition capable of fading out skin defects, such as microreliefs and colour variations, while at the same time retaining the skin's natural appearance due essentially to the translucent nature of this thin film, that is to say of making the skin colour variations and microreliefs, the pores and fine lines less visible.

Thus, another subject of the invention consists of the cosmetic use of the composition as defined above, to fade out imperfections in the skin relief and/or to conceal the microreliefs, wrinkles, fine lines and/or pores of the skin.

A subject of the invention is also the cosmetic use of the composition as defined above, for making up the skin, the eyelashes, the lips and/or the hair.

The invention also relates to a cosmetic process for treating the skin, which is intended to give it a matt appearance and/or to conceal the defects in the skin relief, characterized in that a composition as defined above is applied to the skin.

Other characteristics and advantages of the invention will emerge more clearly on reading the description which follows, given by way of non-limiting illustration.

### DETAILED DESCRIPTION OF EMBODIMENTS

The examples which follow illustrate the preparation of cosmetic compositions with an optical effect according to the invention, comprising polymers comprising specific water-soluble units and units with an LCST.

The polymer used in these examples consists of a polyacrylic acid (PAA) backbone bearing side chains or grafts consisting of specific units with an LCST. It is characterized by the molar mass of the water-soluble backbone (polyacrylic acid), the chemical nature of the chains with an LCST, their proportion by mass in the polymer and their molar mass.

The properties of the polymer of the invention are also compared with those of a polyacrylic acid taken as a comparative reference polymer, which is representative of polymers not bearing side chains with an LCST, which are the polymers included in the cosmetic compositions with an optical effect of the prior art.

The characteristics of the polymer according to the invention and of the (comparative) reference polymer used are given in Table I.

TABLE 1

| | | | Proportion: | |
|---|---|---|---|---|
| | | | units with | Degree |
| | | | an LCST in | of |
| | Water- | | the final | graft- |
| | soluble | Grafts (units | polymer (by | ing |
| | backbone | with an LCST) | weight) | (mol %) |
| Polymer 1 | Polyacrylic | Random | 51% | 3.9% |
| according | acid; | (EO)₆(PO)₃₉ | | |
| to the | MW = 450 000 | Jeffamine | | |
| invention | | M-2005; | | |
| | | MW = 2600 | | |
| Reference | Polyacrylic | / | 0% | 0% |
| polymer 2 | acid; | | | |
| | MW = 450 000 | | | |

These polymers are prepared in the following manner.

### Preparation of Polymer 1 According to the Invention

3 g of polyacrylic acid with a mean molar mass of 450 000 g/mol (Aldrich) are dissolved in 220 ml of N-methylpyrrolidone in a 500 ml reactor equipped with a condenser, with stirring at 60° C. for 12 hours.

4.181 g of monoamino random (EO)₆(PO)₃₉ copolymer with a molar mass of 2600 g/mol having a cloud point, at a concentration of 1% by weight in water, of 16° C. (Jeffamine M-2005 from Huntsman) are dissolved in 50 ml of N-methylpyrrolidone, with stirring at 20° C. for 15 minutes. The solution obtained is added dropwise to the reaction medium containing the polyacrylic acid, with vigorous stirring at 60° C.

2.158 g of dicyclohexylcarbodiimide are dissolved in 30 ml of N-methylpyrrolidone, with stirring at 20° C. for 15 minutes. The solution obtained is added dropwise to the reaction medium containing the polyacrylic acid and the monoamino random (EO)₆(PO)₃₉ copolymer, with vigorous stirring at 60° C. The final mixture is stirred for 12 hours at 60° C.

The mixture is cooled to 20° C. and is then placed in a refrigerator at 4° C. for 24 hours. The crystals of dicyclohexylurea formed are removed by filtration of the reaction medium.

The polymer is then neutralized with 19 g of 35% sodium hydroxide (4-fold excess relative to the number of moles of acrylic acid), leading to its precipitation. After standing for 12 hours, the reaction medium is filtered so as to recover the precipitated polymer. This polymer is dried under vacuum at 35° C. for 24 hours.

13.55 g of solid are recovered and are dissolved in 2 liters of deionized water. This solution is ultrafiltered using a Millipore ultrafiltration system containing a membrane with a cutoff threshold set at 10 000 daltons. The solution thus purified is freeze-dried so as to collect the polymer in solid form.

7.05 g of polyacrylic acid (450 000 g/mol) grafted with 3.9% (on a molar basis) of monoamino random (EO)₆(PO)₃₉ copolymer are obtained.

The proportion by mass of the units with an LCST in the final polymer is 51%.

The polymer thus obtained has a solubility in water, at 20° C., of at least 10 g/l.

The comparative, control, reference polymer 2 is a polymer available from the company Aldrich under the name Acrylic acid of molar mass 450 000 g/mol.

### EXAMPLE 1

In this example, the tack of a film of the reference acrylic acid polymer 2 is studied in comparison with a film of heat-induced gelling polymer 1 according to the invention (acrylic acid polymer containing grafts with an LCST), as a function of the temperature and the relative humidity (RH).

### Procedure

1) Preparation of Films of Each of the Polymers

A solution of the test material (water +polymer) in a volatile solvent (water), at a concentration such that the amount of solution to be dried spreads out satisfactorily in the container and such that the thickness of the deposit is 100 microns, is left to dry in the absence of dust in a metal container with a perfectly rectified base placed on a totally horizontal surface.

2) Measurement of the Tack

After drying for 24 hours at room temperature, the metal container is placed on a plate with water circulation, the thermostatic bath of which is set at 35° C. (the surface of the film will be about 32° C.) for a minimum of one hour.

The feeler (made of aluminium alloy, 6 mm diameter) is placed on the arm of the texture analyser (TATXT2 from Rheo) and, after calibration, it is brought to within about 2 mm of the surface to be measured. It is necessary to ensure, during the test, that the two surfaces to be placed in contact (film/feeler) are parallel. This may be achieved, for example, by placing the mould on an assembly whose horizontality is adjustable. The feeler is moved at a rate of 1 mm/s until in contact, and the feeler pressure is then brought to 10 000 Pa at the speed of 0.1 mm/s. This pressure is maintained for 20 s, followed by returning to the starting position at a speed of 0.1 mm/s. The film tack is evaluated during this retreat phase by measuring the energy required to separate the two surfaces in contact (integration of the “force versus displacement” curve).

### Results

Tack (separation energy) in J/m².

| | Temperature | | |
|---|---|---|---|
| | 27° C. | 30° C. | 35° C. |
| Relative humidity (RH) | 80% RH | 30% RH | 80% RH |
| Tack of the non-grafted | 0.9 ± 0.3 | 0 ± 0.03 | 0.5 ± 0.3 |
| control polymer | | | |
| Tack of the heat-induced | 0 | 0 ± 0.03 | 0 ± 0.03 |
| gelling polymer | | | |

### Conclusions

It emerges from this study that the tack of the non-grafted, control, reference polymer 2 is sensitive to the relative humidity, unlike the grafted polymer 1 according to the invention, whose tack remains zero irrespective of the relative humidity or the temperature under consideration.

### EXAMPLE 2

This example relates to a mascara composition according to the invention comprising the polymer 1 in accordance with the invention.

Mascara

| | |
|---|---|
| Beeswax | 10% |
| Carnauba wax | 10% |
| Stearic acid | 5.6% |
| Triethanolamine | 3.1% |
| Pigments | 5% |
| Polymer of the invention | 6% |
| Demineralized water | 60.3% |

This mascara composition gives deposits or films with good staying power under a humid atmosphere (80% RH).

### EXAMPLE 3

This example relates to a fluid foundation composition comprising the polymer 1 in accordance with the invention.

Fluid foundation (O/W emulsion)

| Oily phase | |
|---|---|
| Parleam oil | 12% |
| Cyclopentadimethylsiloxane | 8% |
| Aqueous phase | |
| Pigments | 10% |
| Polymer of the invention | 8% |
| Preserving agent | 0.2% |
| Demineralized water | 61.8% |

This fluid foundation gives deposits or films with good staying power under a humid atmosphere (80% RH).

### REFERENCES

[1] D. Hourdet et al., Polymer, 1994, Vol. 35, No. 12, pages 2624-2630.

[2] F. L'Alloret et al., Coll. Polym. Sci., 1995, Vol. 273, No. 12, pages 1163-1173.

[3] F. L'Alloret, Revue de l'Institut Francais du Petrole [Review of the French Petroleum Institute], Vol. 52, No. 2, pages 117-128.

[4] EP-A-0 583 814.

[5] EP-A-0 629 649.

[6] EP-A-95 24430.

[7] US-A-5 939 485.

[8] WO-A-97 00275.

[9] WO-A-98 48768.

[10] WO-A-00 35961.

[11] Articles by Taylor et al., Journal of Polymer Science, part A: Polymer Chemistry, 1975, 13, 2551.

[12] J. Bailey et al., Journal of Applied Polymer Science, 1959, 1, 56.

[13] Heskins et al., Journal of Macromolecular Science, Chemistry A2, 1968, 1441.

1. Cosmetic composition comprising an aqueous phase, said aqueous phase comprising at least one compound with an optical effect and a polymer comprising water-soluble units and units having in water a lower critical solution temperature LCST, the heat-induced demixing temperature in aqueous solution of said units with an LCST being from 5 to 40° C. for a concentration by mass in water of from 1% to 25% of said units.

2. Cosmetic composition according to claim 1, in which the heat-induced demixing temperature in aqueous solution of the units with an LCST of the polymer is from 10 to 35° C. for a concentration by mass in water of from 1% to 25% of said units.

3. Cosmetic composition according to claim 1, in which the polymer is in the form of a block polymer comprising blocks consisting of water-soluble units alternating with blocks consisting of units with an LCST; or in the form of a grafted polymer whose backbone is formed from water-soluble units, said backbone bearing grafts consisting of units with an LCST; said polymers possibly being partially crosslinked.

4. Cosmetic composition according to any one of the preceding claims, in which the water-soluble units are totally or partially obtainable by polymerization or by polycondensation, or alternatively consist totally or partially of natural polymers or modified natural polymers.

5. Cosmetic composition according to claim 4, in which the water-soluble units are totally or partially obtainable by polymerization, especially free-radical polymerization, of at least one monomer chosen from the following monomers:
(meth)acrylic acid;
vinyl monomers of formula (I) below: 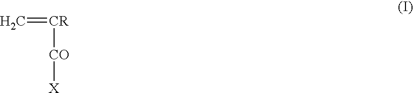
in which:
R is chosen from H. —CH₃, —C₂H₅ or —C₃H₇; and
X is chosen from:
alkyl oxides of —OR′ type in which R′ is a linear or branched, saturated or unsaturated hydrocarbon-based radical containing from 1 to 6 carbon atoms, optionally substituted with at least one halogen atom (iodine, bromine, chlorine or fluorine); a sulphonic (—SO₃⁻), sulphate (—SO₄⁻), phosphate (—PO₄H₂); hydroxyl (—OH); primary amine (—NH₂); secondary amine (—NHR₁), tertiary amine (—NR₁R₂) or quaternary amine (—N⁺R₁R₂R₃) group with R₁, R₂ and R₃ being, independently of each other, a linear or branched, saturated or unsaturated hydrocarbon-based radical containing 1 to 6 carbon atoms, with the proviso that the sum of the carbon atoms of R′+R₁+R₂+R₃ does not exceed 7; and
—NH₂, —NHR₄ and —NR₄R₅ groups in which R₄ and R₅ are, independently of each other, linear or branched, saturated or unsaturated hydrocarbon-based radicals containing 1 to 6 carbon atoms, with the proviso that the total number of carbon atoms of R₄+R₅ does not exceed 7, the said R₄ and R₅ optionally being substituted with a halogen atom (iodine, bromine, chlorine or fluorine); a hydroxyl (—OH); sulphonic (—SO₃⁻); sulphate (—SO₄⁻); phosphate (—PO₄H₂); primary amine (—NH₂); secondary amine (—NHR₁), tertiary amine (—NR₁R₂) and/or quaternary amine (—N⁺R₁R₂R₃) group with R₁, R₂ and R₃ being, independently of each other, a linear or branched, saturated or unsaturated hydrocarbon-based radical containing 1 to 6 carbon atoms, with the proviso that the sum of the carbon atoms of R₄+R₅+R₁+R₂+R₃ does not exceed 7;
maleic anhydride;
itaconic acid;
vinyl alcohol of formula CH₂═CHOH;
vinyl acetate of formula CH₂═CH-OCOCH₃;
N-vinyllactams such as N-vinylpyrrolidone, N-vinylcaprolactam and N-butyrolactam;
vinyl ethers of formula CH₂═CHOR₆ in which R₆ is a linear or branched, saturated or unsaturated hydrocarbon-based radical containing from 1 to 6 carbon atoms;
water-soluble styrene derivatives, especially styrene sulphonate;
dimethyldiallylammonium chloride; and
vinylacetamide.

6. Cosmetic composition according to claim 4, in which the water-soluble units of the polymer consist totally or partially of polycondensates or of natural polymers or modified natural polymers chosen from one or more of the following components:
water-soluble polyurethanes;
xanthan gum;
alginates and derivatives thereof such as propylene glycol alginate;
cellulose derivatives and especially carboxymethylcellulose, hydroxypropylcellulose, hydroxyethylcellulose and quaternized hydroxyethylcellulose;
galactomannans and derivatives thereof, such as Konjac gum, guar gum, hydroxypropylguar, hydroxypropylguar modified with sodium methylcarboxylate groups, and hydroxypropyltrimethylammonium guar chloride; and
polyethyleneimine.

7. Cosmetic composition according to either of claims 5 and 6, in which the water-soluble units of the polymer have a molar mass ranging from 1000 g/mol to 5 000 000 g/mol when they constitute the water-soluble backbone of a grafted polymer, or a molar mass ranging from 500 g/mol to 100 000 g/mol when they constitute a block of a multiblock polymer.

8. Cosmetic composition according to any one of claims 1 to 7, in which the units with an LCST of the polymer consist of one or more polymers chosen from the following polymers:
polyethers such as polyethylene oxide (PEO), polypropylene oxide (PPO) or random copolymers of ethylene oxide (EO) and of propylene oxide (PO);
polyvinyl methyl ethers;
polymeric and copolymeric N-substituted acrylamide derivatives containing units with an LCST; and
polyvinylcaprolactam and vinylcaprolactam copolymers.

9. Cosmetic composition according to any one of claims 1 to 8, in which the units with an LCST of the polymer consist of polypropylene oxide (PPO)ₙ where n is an integer from 10 to 50, or of random copolymers of ethylene oxide (EO) and of propylene oxide (PO), represented by the formula:
(EO)ₘ(PO)ₙ
in which m is an integer ranging from 1 to 40 and preferably from 2 to 20, and n is an integer ranging from 10 to 60 and preferably from 20 to 50.

10. Cosmetic composition according to claim 9, in which the molar mass of the units with an LCST of the polymer is from 500 to 5300 g/mol, preferably from 1500 to 4000 g/mol.

11. Cosmetic composition according to claim 10, in which the units with an LCST of the polymer consist of a polymeric or copolymeric N-substituted acrylamide derivative containing units with an LCST.

12. Cosmetic composition according to claim 11, in which the units with an LCST of the polymer consist of a polymer chosen from poly-N-isopropylacrylamide, poly-N-ethylacrylamide and copolymers of N-isopropylacrylamide or of N-ethylacrylamide and of a vinyl monomer chosen from the monomers having the formula (I) given in claim 5, maleic anhydride, itaconic acid, vinylpyrrolidone, styrene and its derivatives, dimethyldiallylammonium chloride, vinylacetamide, vinyl alcohol, vinyl acetate, vinyl ethers and vinyl acetate derivatives.

13. Cosmetic composition according to claim 11 or 12, in which the molar mass of the units with an LCST of the polymer is from 1000 g/mol to 500 000 g/mol and preferably from 2000 to 50 000 g/mol.

14. Cosmetic composition according to any one of claims 1 to 8, in which the units with an LCST of the polymer consist of a polyvinylcaprolactam or a copolymer of vinylcaprolactam and of a vinyl monomer chosen from the monomers corresponding to formula (I) given in claim 5, maleic anhydride, itaconic acid, vinylpyrrolidone, styrene and its derivatives, dimethyldiallylammonium chloride, vinylacetamide, vinyl alcohol, vinyl acetate, vinyl ethers and vinyl acetate derivatives.

15. Cosmetic composition according to claim 14, in which the molar mass of the units with an LCST is from 1000 to 500 000 g/mol and preferably from 2000 to 50 000 g/mol.

16. Cosmetic composition according to any one of claims 1 to 15, in which the proportion by mass of the units with an LCST of the polymer is from 5 to 70%, preferably from 20 to 65% and better still from 30 to 60% relative to the polymer.

17. Cosmetic composition according to any one of claims 1 to 16, in which the concentration by mass of polymer in the aqueous phase is from 0.1 to 20% and preferably from 0.5 to 10%.

18. Cosmetic composition according to any one of the preceding claims, in which the compound with an optical effect is chosen from fillers, pigments, nacres, tensioning agents and matt-effect polymers, and mixtures thereof.

19. Cosmetic composition according to any one of claims 1 to 18, which is a dispersion.

20. Cosmetic composition according to any one of claims 1 to 18, which is an oil-in-water emulsion comprising, in addition to the aqueous phase, an oily phase dispersed in the aqueous phase.

21. Cosmetic composition according to claim 20, in which the aqueous phase also optionally comprises an emulsifying surfactant.

22. Cosmetic composition according to any one of claims 1 to 21, in which the aqueous phase also optionally comprises a gelling agent in a concentration of from 0.01 to 5% by weight relative to the total weight of the composition.

23. Cosmetic composition according to any one of claims 1 to 22, in which the aqueous phase consists of a physiologically acceptable medium.

24. Cosmetic composition according to any one of claims 1 to 23, which is in the form of a cosmetic, make-up or care composition that may be applied to the skin, including the scalp, the nails, the hair, the eyelashes, the eyebrows, the eyes, mucous membranes and semi-mucous membranes, and any other area of body or facial skin.

25. Composition according to any one of claims 1 to 24, which is chosen from foundations, face powders, eyeshadows, mascaras, products providing coverage or “soft focus”, antisun products and hair products.

26. Use of the polymer as described in claim 1, to eliminate or reduce the tack of a film or deposit obtained from a composition with an optical effect containing the said polymer.

27. Use of the polymer as described according to claim 1, to maintain the staying power of a film or deposit obtained from a composition with an optical effect containing the said polymer.

28. Use according to claim 25 or 26, in which the said film or deposit is exposed to a hot and/or humid atmosphere.

29. Use according to claim 27, in which the relative humidity (RH) of the said atmosphere is from 40 to 95%.

30. Use according to claim 27 or 28, in which the temperature of the said atmosphere is from 25 to 45° C.

31. Cosmetic use of the composition according to any one of claims 1 to 25, to fade out imperfections in the skin relief and/or to conceal the microreliefs, wrinkles, fine lines and/or pores of the skin.

32. Cosmetic use of the composition according to any one of claims 1 to 25, for making up the skin, the eyelashes, the lips and/or the hair.

33. Cosmetic process for treating the skin, which is intended to give it a matt appearance and/or to conceal the defects in the skin relief, characterized in that the composition according to any one of claims 1 to 25 is applied to the skin.
